(19) United States
(12) Patent Application Publication
(10) Pub. No.: US 2026/0334792 A1
(43) Pub. Date: Oct. 29, 2026

(54) MULTI-FIBER CATHETER PROBE FOR MULTI-PARAMETRIC OPTICAL COHERENCE TOMOGRAPHY

(71) Applicant: The General Hospital Corporation, Boston, MA (US)

(72) Inventors: Taylor Cannon, Boston, MA (US); Milen Shishkov, Boston, MA (US); Brett E. Bouma, Boston, MA (US); Néstor Uribe-Patarroyo, Boston, MA (US); Martin Villiger, Boston, MA (US)

(21) Appl. No.: 19/112,012

(22) PCT Filed: Dec. 8, 2023
(86) PCT No.: PCT/US2023/083182

§ 371 Date: Mar. 14, 2025

Related U.S. Application Data
(60) Provisional application No. 63/386,670, filed on Dec. 8, 2022.

(51) Int. Cl. A61B 5/00

(52) U.S. Cl. CPC A61B 5/0066; A61B 5/0084; A61B 5/7203; A61B 2562/0233

(57) ABSTRACT

A multi-resolution OCT imaging system for improved tissue scattering property characterization. The system includes a probe with one or more optical fibers and optical elements for scanning a sample with two or more beams. A single fiber probe includes a diffractive element for splitting a single light beam from the fiber optic into two or more beams. The multiple beams entering and reflecting from the tissue differ in path length and numerical aperture (NA) to provide different resolution imaging for determining multiple scattering in the tissue.

### CROSS REFERENCE TO RELATED APPLICATIONS

The present application is based on, claims priority to, and incorporates herein by reference in its entirety for all purposes, U.S. Provisional Application Ser. No. 63/386,670, filed Dec. 8, 2022.

### STATEMENT REGARDING FEDERALLY SPONSORED RESEARCH OR DEVELOPMENT

This invention was made with government support under award numbers 5K25EB024595-04, R01EB03330, and 2P41EB015903-11A1 from the NIH-NIBIB. The government has certain rights in the invention.

### BACKGROUND

Endoscopic visualization of tissue at a microstructural (e.g., nuclei-level features) is challenging but there is a high motivation to do so given the importance of evaluating the sizes and densities of nuclei in epithelia as a hallmark of dysplasia. However, the resolution of most endoscopic OCT systems is still limited to tens of microns, thus the characterization of nuclei within tissue remains a challenge. High-resolution OCT systems have been demonstrated for endoscopic imaging, but hardware and data processing challenges limit the volumes that can be characterized, and greatly complicate the pathway towards clinical adoption of these devices.

### SUMMARY

To address one or more of the drawbacks of known systems, novel catheter-based probes for endoscopic OCT imaging are disclosed which simultaneously acquire OCT images at two different resolutions, separated in a single acquired tomogram (cross-sectional image) in depth. This is accomplished using single-or multi-fiber probes for sending two or more focused beams of different effective numerical aperture (NA) through a single output lens and into tissue at a slight offset.

In a first embodiment, the device includes a single optical fiber along with free-space, spatial-multiplexing elements, enclosed in a catheter sheath compatible for insertion into an endoscope instrument channel, terminating at one end with optical components (e.g., diffractive element, lenses, and mirrors) for beam splitting, focusing, and directing the light that passes through them. The free-space spatial-multiplexing element could function as a diffraction grating to generate at least two diffraction orders differing in angle, or a lateral displacement beam splitter. At the other end, the fiber is connected within a 3D-printed handle containing mechanical elements to stabilize the fiber as it is spun. This end, via the handle, interfaces with the imaging console both mechanically to connect the fiber to the rotary junction to actuate spinning and circumferential image formation, and also optically to connect the fiber to the light source contained within the console.

In a second embodiment, the device includes two or more optical fibers enclosed in a catheter sheath compatible for insertion into an endoscope instrument channel, terminating at one end with additional optical components (e.g., lenses and mirrors) for focusing and directing the light that passes through them. At the other end, the fibers are connected within a 3D-printed handle containing a beam splitter and mechanical elements to stabilize the fibers as they are spun. This end, via the handle, interfaces with the imaging console both mechanically to connect the fibers to the rotary junction to actuate spinning and circumferential image formation, and also optically to connect the fibers to the light source contained within the console.

According to one aspect, an apparatus for multi-parametric optical coherence tomography (OCT) is provided, the apparatus including: an imaging system; a probe coupled to the imaging system and including a proximal end and a distal end, the probe including: at least one optical fiber including a fiber proximal end and a fiber distal end and disposed within the probe, the fiber distal end extending at least a partial length between the proximal end and the distal end of the probe, and a plurality of optical elements positioned at the distal end of the probe and positioned distal to the fiber distal end; and a processor coupled to the imaging system and configured to: direct at least two spatially-offset light beams at a sample, collect data returned from the sample from the at least two light beams through the probe, combine the data, and analyze the combined data to determine at least one sample parameter.

In some embodiments of the apparatus, the plurality of optical elements may include a lens and a reflective element configured to direct a beam from the at least one optical fiber into a sample, wherein the reflective element may be disposed distal to the lens. In other embodiments of the apparatus, the lens may be a graded refractive index (GRIN) lens. In certain embodiments of the apparatus, the reflective element may be a mirror. In particular embodiments of the apparatus, the plurality of optical elements may further include a diffractive optical element. In some embodiments of the apparatus, the diffractive optical element may include a beam splitter.

In various embodiments of the apparatus, the at least two spatially-offset light beams may include a first light beam having a first numerical aperture (NA₁) and a second light beam having a second numerical aperture (NA₂) different from the first numerical aperture (NA₁). In some embodiments of the apparatus, the first numerical aperture may be higher than the second numerical aperture. In particular embodiments of the apparatus, the data may include a first tomogram including data from the first beam and a second tomogram including data from the second beam. In certain embodiments of the apparatus, the first tomogram and the second tomogram may include a single tomogram.

In particular embodiments of the apparatus, the processor, when combining the data, may be further configured to: subtract noise from the first tomogram and the second tomogram, separate the first tomogram and second tomogram, compensate for a confocal function of the imaging system, and de-speckle the first tomogram and the second tomogram.

In some embodiments of the apparatus, the imaging system may include an optical interferometric system. In particular embodiments of the apparatus, the optical interferometric system may include a spectral domain optical coherence tomography (SD-OCT) system or an optical frequency domain imaging (OFDI) system, where the probe may be an OCT probe. In various embodiments of the apparatus, the OCT probe may be a rotary probe coupled to the optical interferometric system by a rotary junction, and the processor, when collecting the data, may be further configured to: cause the OCT probe to rotate, collect the data from the optical interferometric system at a plurality of radial positions, and generate a cross-sectional image of the sample based on collecting data from the optical interferometric system at the plurality of radial positions. In some embodiments of the apparatus, the processor, when analyzing the data, may be further configured to: calculate attenuation coefficients (u) for each of the first tomogram and the second tomogram, and determine a degree of multiple scattering in the sample based on calculating the attenuation coefficients.

In some embodiments of the apparatus, the probe may include at least two optical fibers. In certain embodiments of the apparatus, the at least two optical fibers may differ from one another in at least one of: output beam diameter, fiber length, or type of functional tip at the fiber distal end. In particular embodiments of the apparatus, the at least two optical fibers may include a first optical fiber and a second optical fiber, where the first optical fiber may be adjacent to the second optical fiber, the at least two spatially-offset light beams may include a first light beam and a second light beam, and the first optical fiber may emit the first light beam having a first numerical aperture and the second optical fiber may emit the second light beam having a second numerical aperture different from the first numerical aperture.

In various embodiments of the apparatus, the first optical fiber may include a first optical element coupled to a distal end thereof and configured to emit the first light beam with the first numerical aperture, and the second optical fiber may include a second optical element coupled to a distal end thereof and configured to emit the second light beam with the second numerical aperture. In particular embodiments of the apparatus, the first optical fiber and the second optical fiber may be different lengths. In certain embodiments of the apparatus, the processor, when directing at least two spatially-offset light beams at the sample, may be further configured to: direct the first light beam from the first optical fiber at the sample and direct the second light beam from the second optical fiber at the sample, and the processor, when collecting data returned from the sample, may be further configured to: collect data returned from the sample based on the first light beam and the second light beam, and the processor, when combining the data, may be further configured to: combine the data returned from the sample based on the first light beam and the second light beam to form a single tomogram.

In various embodiments of the apparatus, the processor, when combining the data, may be further configured to: combine the data to provide a depth-multiplexed optical signal based on at least one of a spectral, polarization, angular, or focal variation between the at least two spatially-offset light beams.

In certain embodiments of the apparatus, the plurality of optical elements may include a free-space spatial multiplexing element. In some embodiments of the apparatus, the free-space spatial multiplexing element may be configured to split a light beam into the at least two spatially-offset light beams. In particular embodiments of the apparatus, a first path length of a first light beam of the at least two spatially-offset light beams may be shorter than a second path length of a second light beam of the at least two spatially-offset light beams. In some embodiments of the apparatus, a first numerical aperture of a first light beam of the at least two spatially-offset light beams may be higher than a second numerical aperture of a second light beam of the at least two spatially-offset light beams. In various embodiments of the apparatus, the free-space spatial multiplexing element may include at least one of a lateral beam splitter or a diffraction grating combined with a lens.

According to another aspect, a method for multi-parametric optical coherence tomography (OCT) is provided, the method including: providing a probe coupled to an imaging system, the probe including a proximal end and a distal end, the probe including: at least one optical fiber including a fiber proximal end and a fiber distal end and disposed within the probe, the fiber distal end extending at least a partial length between the proximal end and the distal end of the probe, and a plurality of optical elements positioned at the distal end of the probe and positioned distal to the fiber distal end; and directing, using a processor coupled to the imaging system, at least two spatially-offset light beams at a sample, collecting, using the processor, data returned from the sample from the at least two light beams through the probe, combining, using the processor, the data, and analyzing, using the processor, the combined data to determine at least one sample parameter.

In various embodiments of the method, the plurality of optical elements may include a lens and a reflective element, where the reflective element may be disposed distal to the lens, and where the method may further include: directing a beam from the at least one optical fiber into a sample via the lens and the reflective element. In some embodiments of the method, the lens may be a graded refractive index (GRIN) lens. In certain embodiments of the method, the reflective element may be a mirror. In particular embodiments of the method, the plurality of optical elements may further include a diffractive optical element. In some embodiments of the method, the diffractive optical element may include a beam splitter.

In certain embodiments of the method, directing at least two spatially-offset light beams at a sample may further include: directing at least two spatially-offset light beams at a sample, where the at least two spatially-offset light beams may include a first light beam having a first numerical aperture (NA₁) and a second light beam having a second numerical aperture (NA₂) different from the first numerical aperture (NA₁). In some embodiments of the method, the first numerical aperture may be higher than the second numerical aperture. In particular embodiments of the method, collecting data may further include: collecting a first tomogram including data from the first beam and a second tomogram comprising data from the second beam. In particular embodiments of the method, the first tomogram and the second tomogram may include a single tomogram. In particular embodiments of the method, combining the data may further include: subtracting noise from the first tomogram and the second tomogram, separating the first tomogram and second tomogram, compensating for a confocal function of the imaging system, and de-speckling the first tomogram and the second tomogram.

In some embodiments of the method, providing a probe coupled to an imaging system may further include: providing the probe coupled to the imaging system, where the imaging system may include an optical interferometric system. In particular embodiments of the method, the optical interferometric system may include a spectral domain optical coherence tomography (SD-OCT) system or an optical frequency domain imaging (OFDI) system, where the probe may be an OCT probe. In various embodiments of the method, the OCT probe may be a rotary probe coupled to the optical interferometric system by a rotary junction, and collecting the data may further include: causing the OCT probe to rotate, collecting the data from the optical interferometric system at a plurality of radial positions, and generating a cross-sectional image of the sample based on collecting data from the optical interferometric system at the plurality of radial positions.

In particular embodiments of the method, analyzing the data may further include: calculating attenuation coefficients (μ) for each of the first tomogram and the second tomogram, and determining a degree of multiple scattering in the sample based on calculating the attenuation coefficients.

In various embodiments of the method, the probe may include at least two optical fibers. In some embodiments of the method, the at least two optical fibers may differ from one another in at least one of: output beam diameter, fiber length, or type of functional tip at the fiber distal end. In certain embodiments of the method, the at least two optical fibers may include a first optical fiber and a second optical fiber, where the first optical fiber may be adjacent to the second optical fiber, the at least two spatially-offset light beams may include a first light beam and a second light beam, and the method may further include: emitting the first light beam having a first numerical aperture from the first optical fiber, and emitting the second light beam having a second numerical aperture from the second optical fiber, where the second numerical aperture may be different from the first numerical aperture.

In particular embodiments of the method, the first optical fiber may include a first optical element coupled to a distal end thereof, where the second optical fiber may include a second optical element coupled to a distal end thereof, and the method may further include: emitting the first light beam with the first numerical aperture from the first optical element, and emitting the second light beam with the second numerical aperture from the second optical element. In various embodiments of the method, the first optical fiber and the second optical fiber may be different lengths.

In some embodiments of the method, directing at least two spatially-offset light beams at the sample may further include: directing the first light beam from the first optical fiber at the sample and directing the second light beam from the second optical fiber at the sample, where collecting data returned from the sample may further include: collecting data returned from the sample based on the first light beam and the second light beam, and where combining the data may further include: combining the data returned from the sample based on the first light beam and the second light beam to form a single tomogram.

In various embodiments of the method, combining the data may further include: combining the data to provide a depth-multiplexed optical signal based on at least one of a spectral, polarization, angular, or focal variation between the at least two spatially-offset light beams.

In some embodiments of the method, the plurality of optical elements may include a free-space spatial multiplexing element. In various embodiments, the method may further include splitting a light beam into the at least two spatially-offset light beams using the free-space spatial multiplexing element. In particular embodiments of the method, a first path length of a first light beam of the at least two spatially-offset light beams may be shorter than a second path length of a second light beam of the at least two spatially-offset light beams. In certain embodiments of the method, a first numerical aperture of a first light beam of the at least two spatially-offset light beams may be higher than a second numerical aperture of a second light beam of the at least two spatially-offset light beams. In particular embodiments of the method, the free-space spatial multiplexing element may include at least one of a lateral beam splitter or a diffraction grating combined with a lens.

According to another aspect, an apparatus for multi-parametric optical coherence tomography (OCT) is provided, the apparatus including: a probe including a proximal end and a distal end, the probe including: at least one optical fiber including a fiber proximal end and a fiber distal end and disposed within the probe, the fiber distal end extending at least a partial length between the proximal end and the distal end of the probe, and a plurality of optical elements positioned at the distal end of the probe and positioned distal to the fiber distal end, where the probe is configured to: direct at least two spatially-offset light beams at a sample, and receive image information returned from the sample from the at least two light beams through the probe.

In various embodiments of the apparatus, the plurality of optical elements may include a lens and a reflective element configured to direct a beam from the at least one optical fiber into a sample, where the reflective element may be disposed distal to the lens. In some embodiments of the apparatus, the lens may be a graded refractive index (GRIN) lens. In particular embodiments of the apparatus, the reflective element may be a mirror. In certain embodiments of the apparatus, the plurality of optical elements may further include a diffractive optical element. In some embodiments of the apparatus, the diffractive optical element may include a beam splitter.

In particular embodiments of the apparatus, the at least two spatially-offset light beams may include a first light beam having a first numerical aperture (NA₁) and a second light beam having a second numerical aperture (NA₂) different from the first numerical aperture (NA₁). In some embodiments of the apparatus, the first numerical aperture may be higher than the second numerical aperture.

In certain embodiments of the apparatus, the image information may include a first tomogram including image information from the first beam and a second tomogram including image information from the second beam. In particular embodiments of the apparatus, the first tomogram and the second tomogram may include a single tomogram.

In various embodiments of the apparatus, the probe may be an OCT probe. In certain embodiments of the apparatus, the probe may include at least two optical fibers. In particular embodiments of the apparatus, the at least two optical fibers may differ from one another in at least one of: output beam diameter, fiber length, or type of functional tip at the fiber distal end. In some embodiments of the apparatus, the at least two optical fibers may include a first optical fiber and a second optical fiber, where the first optical fiber may be adjacent to the second optical fiber, the at least two spatially-offset light beams may include a first light beam and a second light beam, and the first optical fiber may emit the first light beam having a first numerical aperture and wherein the second optical fiber may emit the second light beam having a second numerical aperture different from the first numerical aperture.

In some embodiments of the apparatus, the first optical fiber may include a first optical element coupled to a distal end thereof which may be configured to emit the first light beam with the first numerical aperture, and the second optical fiber may include a second optical element coupled to a distal end thereof which may be configured to emit the second light beam with the second numerical aperture. In various embodiments of the apparatus, the first optical fiber and the second optical fiber may have different lengths.

In particular embodiments of the apparatus, the probe, when directing at least two spatially-offset light beams at the sample, may be further configured to: direct the first light beam from the first optical fiber at the sample and direct the second light beam from the second optical fiber at the sample, and the probe, when receiving image information returned from the sample, may be further configured to: collect image information returned from the sample based on the first light beam and the second light beam.

In some embodiments of the apparatus, the plurality of optical elements may include a free-space spatial multiplexing element. In particular embodiments of the apparatus, the free-space spatial multiplexing element may be configured to split a light beam into the at least two spatially-offset light beams. In certain embodiments of the apparatus, a first path length of a first light beam of the at least two spatially-offset light beams may be shorter than a second path length of a second light beam of the at least two spatially-offset light beams. In various embodiments of the apparatus, a first numerical aperture of a first light beam of the at least two spatially-offset light beams may be higher than a second numerical aperture of a second light beam of the at least two spatially-offset light beams. In some embodiments of the apparatus, the free-space spatial multiplexing element may include at least one of a lateral beam splitter or a diffraction grating combined with a lens.

### BRIEF DESCRIPTIONS OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

FIG. 1A is a schematic of a single fiber probe according to aspect of the present. disclosure. The fiber tip (102) could be fixed proximal to a free-space spatial multiplexing element (104), a focusing lens (106), and mirror (108). Arrows indicate direction towards OCT system and interferometer. The inset on the right is a head-on view down the catheter to view how the two beams are offset from one another.

FIG. 1B is a schematic perspective view of the diffractive optical element of FIG. 1A.

FIG. 1C is a schematic of another single fiber probe according to aspects of the present disclosure.

FIG. 1D is a schematic of a dual-fiber probe according to aspects of the present disclosure which features two optical fibers. The tips of each of the fibers (118, 120) are disposed parallel to one another and proximal to a lens (106) (e.g., a GRIN lens) and a deflecting mirror (108).

FIG. 2 is a series of plots of the effect of solutions of particles of variable size, ranging from predominantly singly scattering behavior to multiply scattering behavior on the comparison of calculated attenuation coefficients (μ) at varying system numerical apertures (NA=0.1-0.3) to a u measured at NA=0.1 (μ:μ(NA=0.1)), where the solutions were imaged using OCT. The reduction in u with increasing NA for larger particles demonstrates the ability to infer multiple scattering behavior from these multi-NA measurements.

FIG. 3A is an optical diagram of a realization of the dual-resolution probe featuring two optical fibers connected via a 50/50 beam splitter, using functionalized two single-mode fiber tips with short (<1 mm) lengths of graded refractive index (GRIN) and coreless fiber to differentially expand and focus the beam. Although the optical pathways are shown separately, they are disposed adjacent to one another in the probe.

FIG. 3B is a schematic of the overhead view of the low-resolution channel and high resolution channel passing into the GRIN lens with lateral offset.

FIG. 4A is a schematic of an endoscope inserted into the of the esophagus.

FIG. 4B is a schematic showing tissue imaging with a rotating catheter probe inserted directly into the esophageal lumen through the endoscope accessory channel, with insufflation of the esophagus to collapse the lumen and increase tissue contact (lower dashed lines showing the collapsed configuration).

FIG. 4C is a schematic showing tissue imaging with a rotating catheter probe inserted into a balloon inflated within the esophagus.

FIG. 5A is a schematic of the fiber configuration of a handle of the dual-fiber probe device.

FIG. 5B is a diagram of a custom handle 500 enclosing the fibers.

FIG. 6 is a schematic of the rotary junction and its connection to various components of a handle and probe.

FIG. 7 is a schematic of an OCT system to be used with aspects of the present disclosure.

FIG. 8A is a schematic of the distal tip of the probe device featuring the two optical fibers fixed parallel to each other and mounted optical elements to generate two beams separated laterally (δx). Proximally, a difference in the lengths of each fiber creates an axial separation of the two output images (δz) in the tomogram of FIG. 8B.

FIG. 8B shows two images in a single tomogram, with each image corresponding to the signal acquired by each fiber as the probe is rotated with respect to the sample surface.

FIG. 9A shows dual-resolution OCT images of a tissue acquired in vivo with the custom probe prototype, with finer resolution visible in the high-NA image copy on top with respect to larger-speckle, lower-resolution image copy on the bottom.

FIG. 9B is a plot of the quantitative analysis of signal profile in an imaging phantom assessed minimum resolvable spot size, wo, as calculated from the measured Rayleigh range.

FIG. 10A is a tomogram showing an artifact.

FIG. 10B is a tomogram after post-processing to correct the image of FIG. 10A with the artifact removed.

### DETAILED DESCRIPTION

Improving the sensitivity of endoscopic optical coherence tomography (OCT) to pre-cancerous esophageal lesions stands to improve patient outcomes. Although nuclei-level features relevant to dysplasia diagnoses are below conventional OCT systems' resolution, tissue scattering properties report local particle size and concentration, indirectly accessing nuclei diameter and nuclear-cytoplasmic ratio (NCR). Recently, we have shown that acquiring co-registered images of a sample at different numerical aperture (NA) enhances scattering property measurements through angular diversity. We present results for a custom-designed, catheter-based, dual-NA endoscopic probe demonstrating improved characterization of phantoms and tissue with histological validation, and potential utility for more sensitive dysplasia diagnoses.

Endoscopic visualization of tissue at a microstructural (e.g., nuclei-level features) is challenging but there is a high motivation to do so given the importance of evaluating the sizes and densities of nuclei in epithelia as a hallmark of dysplasia. This preliminary disease stage is characterized by abnormal morphology of cell nuclei in the tissue epithelium, and specifically, by increases in their diameters and the fraction of the total cell area they occupy (nuclear-cytoplasmic ratio). These microscale changes (less than ~10 μm) are identified using high-resolution digital pathology images obtained after endoscopic tissue biopsy and standard histological processing. Conventional white light endoscopes currently deployed in clinical practice are limited both by their millimeter-level resolution and their inability to visualize below the surface of a tissue. Alternatively, Optical Coherence Tomography (OCT), a high-speed, light-based imaging modality, interrogates sub-surface properties of samples to produce cross-sectional and volumetric images of tissue structure, which have shown promise in visualizing disorders of the gastrointestinal tract when this technique is deployed endoscopically. However, the resolution of most endoscopic OCT systems is still limited to tens of microns, thus the characterization of nuclei within tissue remains a challenge. High-resolution OCT systems have been demonstrated for endoscopic imaging, but hardware and data processing challenges limit the volumes that can be characterized, and greatly complicate the pathway towards clinical adoption of these devices.

As an alternative to direct visualization of nuclei, local changes in their sizes and concentrations can be mapped as a function of how quickly nuclei, acting as light scattering particles, attenuate the light that penetrates into tissue. This metric, the attenuation coefficient (u), is directly related to these physical tissue properties and can be calculated from OCT intensity images. However, challenges in measurement and interpretation of attenuation coefficients in tissue degrade the accuracy and limit the clinical utility of this metric. A complication in measurement is the impact of the OCT system design on the intensity decay in a sample independent of the sample's physical properties, and chiefly the confocal function, a result of beam focusing within a sample, which alters the distribution of intensities about the focus.

Another complication in metric interpretation is the incidence of multiple scattering (MS) of light in tissue, which is not accounted for in theoretical models connecting attenuation coefficient to physical sample properties and makes areas dominated by MS unable to yield accurate particle sizes or concentrations.

Endoscopic Optical Coherence Tomography (OCT) systems stand to benefit from greater quantitative assessment of tissue in clinical settings, such as through the characterization throughout imaged regions of the attenuation coefficient, a metric directly related to the sub-resolution sizes and concentrations of scattering particles (e.g., nuclei) in tissue. However, practical challenges degrade the accuracy and limit the utility of OCT-based attenuation coefficient measurements in clinical scenarios where they could otherwise have impact, such as the diagnosis of dysplasia.

The present disclosure provides catheter-based imaging probe(s) for endoscopic OCT which collect simultaneous OCT images at two separate and customizable resolutions. This strategy mitigates challenges in attenuation coefficient measurement and interpretation to yield a metric more accurately representative of physical properties of an imaged sample.

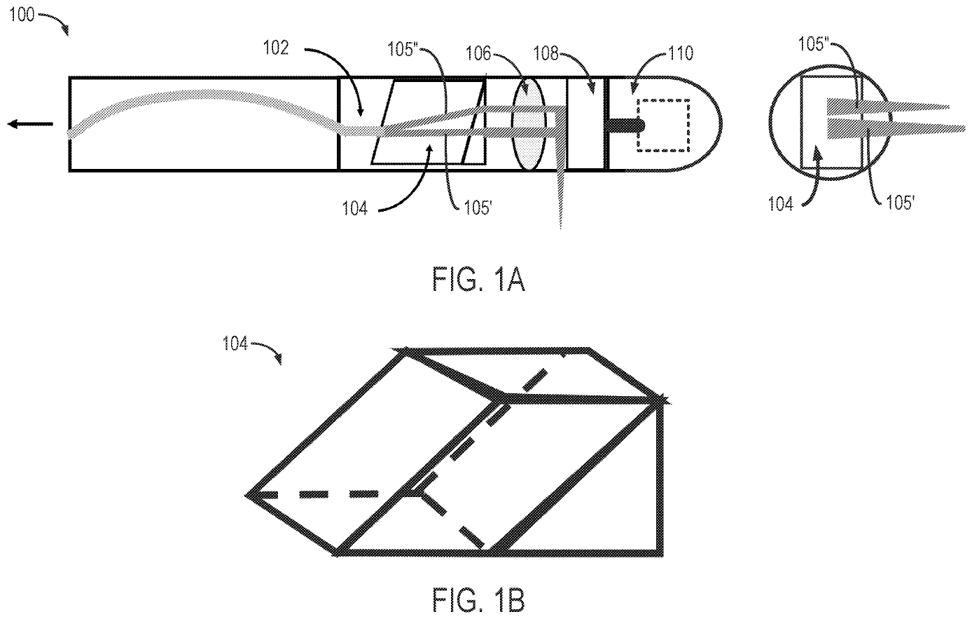

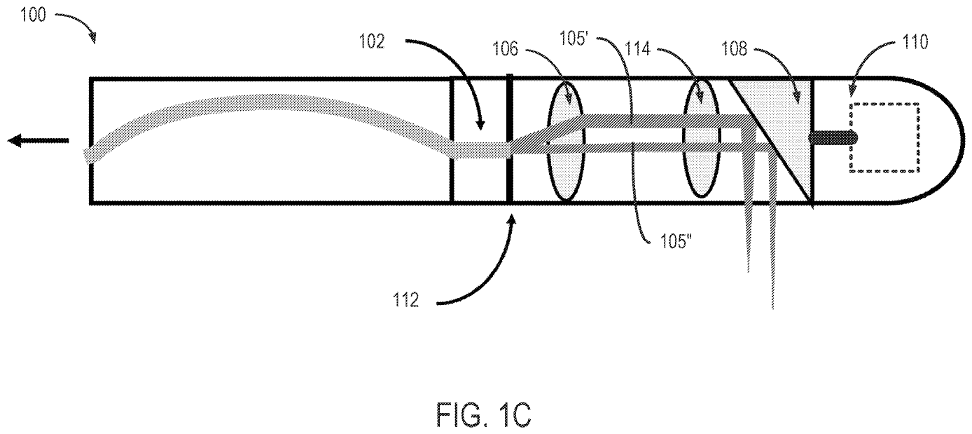

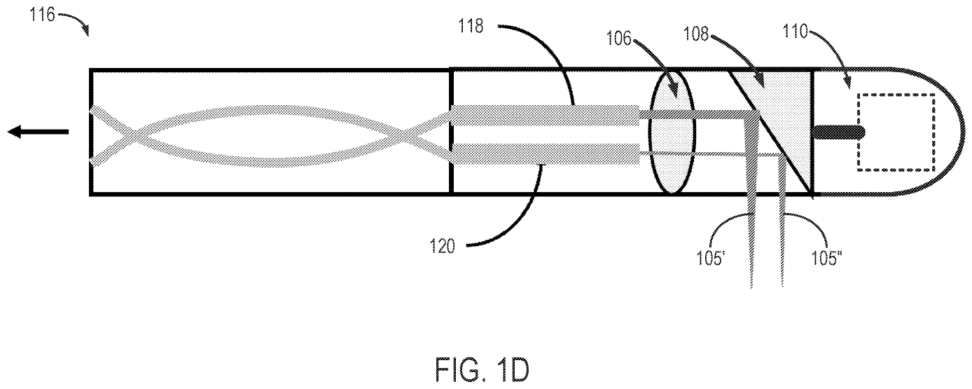

Accordingly, disclosed herein are optical imaging probes to be deployed in a catheter during endoscopic imaging procedures. As illustrated in FIGS. 1A-1C, the probe may contain one or more optical fibers and additional optical elements within the catheter sheath. Referring to FIG. 1A, a distal end of a probe 100 is described, where the inset on the right is a head-on view down the catheter to view how the two beams 105′, 105″ are offset from one another. A single optical fiber 102 with a fiber proximal end and a fiber distal end extends at least partially along the length of the probe 100, terminating in a distal portion thereof. The optical fiber 102 emits a beam of light that passes through a plurality of optical elements. The beam first passes through a diffractive optical element 104 that is configured to split the single beam into two or more non-overlapping beams 105′, 105″. The diffractive optical element 104 may split the single beam into two equivalent beams or may produce two unequal beams in which more of the light goes to one beam than another.

In a non-limiting example, the diffractive optical element 104 includes a diffraction grating to generate at least two diffraction orders differing in angle, and in another non-limiting example the diffractive optical element 104 includes a lateral displacement beam splitter. Based on the properties of the diffractive optical element 104 (which may be made of glass/optical-grade polymer in one non-limiting example) the path traveled through will differ in angle for either of the various different diffraction orders (or the lateral separation of the beams), and as a result each of the spatially-separated beam 105′, 105″ will travel through a different amount of the element material versus air. Since the diffractive optical element 104 material has a higher refractive index than air, this will result in a longer optical distance traveled for the beam that passes through a greater distance within the diffractive optical element 104, which is functionally equivalent to transmitting the beams through fibers having different fiber lengths; as disclosed herein, the differences in path lengths of the beams causes the images from each beam to be shifted in the output tomogram, which permits each image to be separately analyzed. Further, the greater path length that is traveled by one of the beams leads to more divergence of the beam 105′ exiting the fiber, along that distance, where the greater divergence of one of the beams gives it a higher NA than the other, less divergent beam.

Thereafter the two or more beams pass through a lens 106. In a non-limiting example, the lens 106 is a graded refractive index lens. In a non-limiting example a second lens (not shown) may be positioned adjacent to lens 106 to enhance the divergence of the of the two beams 105′, 105″, thereby amplifying the differences in NA between the beams. The beams 105′, 105″ then impinge on a reflective element 108 to change the direction of the beams into the sample. In a non-limiting example, the reflective element 108 may include a mirror.

In a non-limiting example, an optional distal end micromotor 110 may be used to rotate the reflective element 108 in FIG. 1A and FIGS. 1C-1D, while the remaining components of the probe 100 may remain stationary. Alternatively, the probe 100 may be rotated using a rotary junction as will be described in further detail below.

FIG. 1B shows an enlarged view of the diffractive optical element 104 used in FIG. 1A.

Referring to FIG. 1C, an alternative single fiber probe 100 is provided, sharing many of the same elements as in FIG. 1A. A single optical fiber 102 with a fiber proximal end and a fiber distal end extends at least partially along the length of the probe 100, terminating at or near a distal 18 portion of the probe. The optical fiber 102 emits a beam of light that passes through a plurality of optical elements. The beam first passes through a diffractive grating 112 that is configured to split the single beam into two or more non-overlapping beams 105′, 105″. Each of the beams 105′, 105″ will have different paths through the grating, resulting in different path lengths as well as different amounts of divergence, with the beam having traveled further having the greater divergence (and hence higher NA) compared to the other beam. Thereafter the two or more beams 105′, 105″ pass through a lens 106, as previously described. The beams 105′, 105″ then pass through a second lens 114 to enhance the divergence between the two beams. The beams 105′, 105″ then impinge on a reflective element 108 to change the direction of the beams into the sample. In a non-limiting example, the reflective element 108 include a mirror.

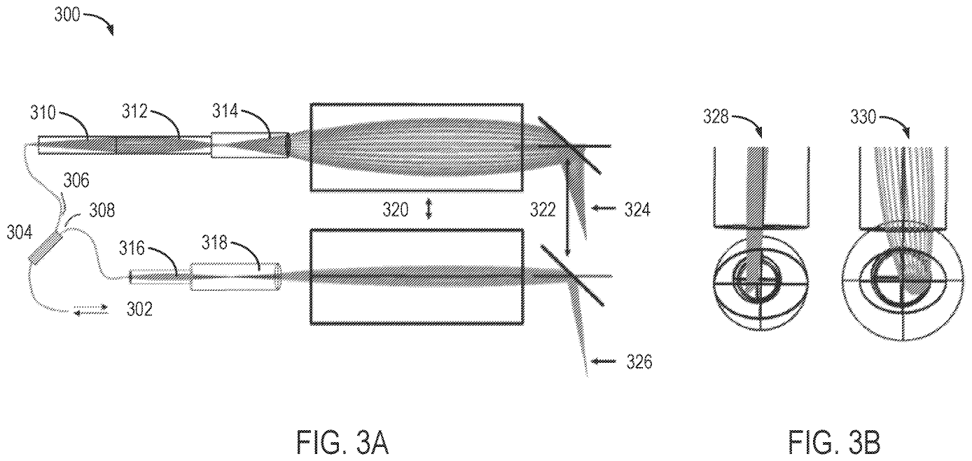

In another embodiment, FIG. 1D illustrates a dual fiber probe 116 with two or more optical fibers. As shown in FIG. 1D, probe 116 includes a first optical fiber 118 and a second optical fiber 120. It is noted that the probe may include any number of optical fibers greater than two, depending on the constraints of the probe design for a specific application. As above with FIG. 1A and FIG. 1C, the first and second optical fibers 118, 120 each have a fiber proximal end and a fiber distal end extending at least partially along the length of the probe 116, terminating in a distal portion thereof. The optical fibers 118, 120 each emit a beam of light 105′, 105″ that passes through a lens 106 and a reflective element 108 to change the direction of the beams into the sample. The light exiting each of the optical fibers 118, 120 passes through various optical elements which produce more or less divergence of the exiting beam to generate the respective NAs of the beams, for example as shown in FIG. 3A and described in the accompanying text below.

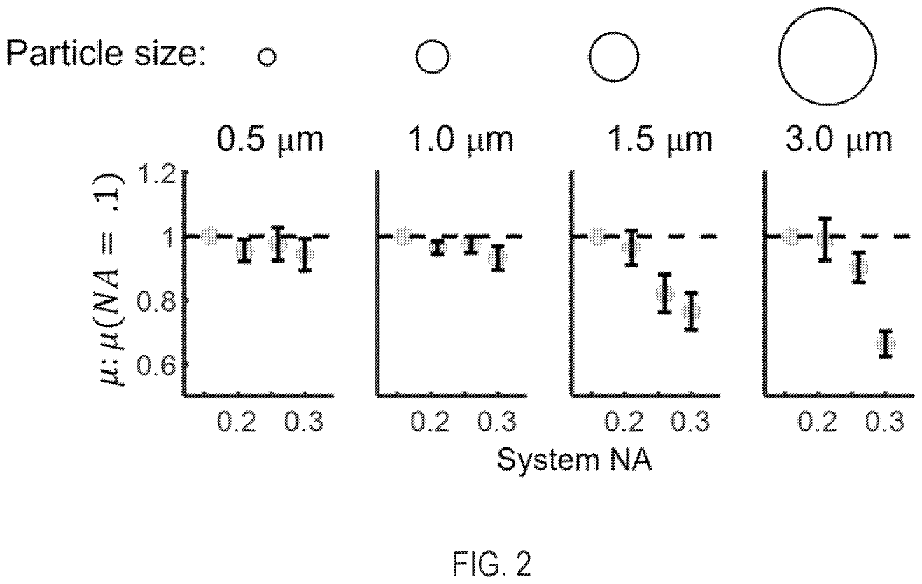

In FIG. 1A and FIG. 1C-1D, the arrow pointing away from the probe at the proximal end (left side of each drawing) indicates a direction towards an OCT system and interferometer. The optical elements described in FIGS. 1A-1D have the effect of adjusting the NA, and thereby the angular diversity, of the beams entering the sample to differentially sample the tissue scattering phase function. It is known that multiple scattering (MS) can impact OCT data, effectively reducing calculated u and hindering their interpretation based on Mie theory. Similar μ(z) values measured at each NA in a region of interest (ROI) indicate minimal MS impact in that region, whereas the more the μ(z) values diverge, the greater the MS impact is (FIG. 2). As shown in FIG. 2, experimentally measured μ(z) in polystyrene bead phantoms compared against theoretical estimates of u calculated with Mie theory reveal that in phantoms with small particles (0.5 μm), u changed minimally with respect to the OCT system NA under which they were measured (with respect to value measured at NA=0.1), but with larger particles (3 μm), μ measurements at each NA deviated both from theoretical values and from each other at increasingly high NA. In a non-limiting example, based on a lower NA channel similar to conventional endoscopic resolution (~30 μm, or NA≈0.06), setting a target for the higher NA channel of a factor of 2-3 higher (NA≈0.1-0.15) balances signal quality and depth-of-focus tradeoffs.

FIGS. 3A-3B demonstrate how target NA values are achieved in a dual fiber catheter. Light from an OCT system 302 enters a 50/50 beam splitter 304 producing two optical fibers 306, 308. In the high NA fiber 306, a section of coreless fiber 310 is followed by GRIN fiber 312 and another segment of coreless fiber 314 to expand the beam. In the low NA fiber 308, a section of coreless fiber 316 is spliced to a section of GRIN fiber 318. Beams exiting elements 314 and 318 pass through a common GRIN lens 320 prior to deflection by a common mirror 322 to send the high NA output beam 324 and low NA output beam 326 into a sample, with scattered imaging signal collected back along the same path. In a non-limiting example, the fibers are slightly different in length (to create different path lengths) to achieve depth multiplexing of each sample arm signal and thus signal separation during simultaneous illumination. Alternatively or additionally, the fiber tips may vary in shape and/or functionalization, although generally the functionalized fiber tips are oriented side-by-side and focused into the same GRIN lens. FIG. 3B shows an overhead view of the low NA channel 328 and high NA channel 330 passing into the GRIN lens 320 with a lateral offset.

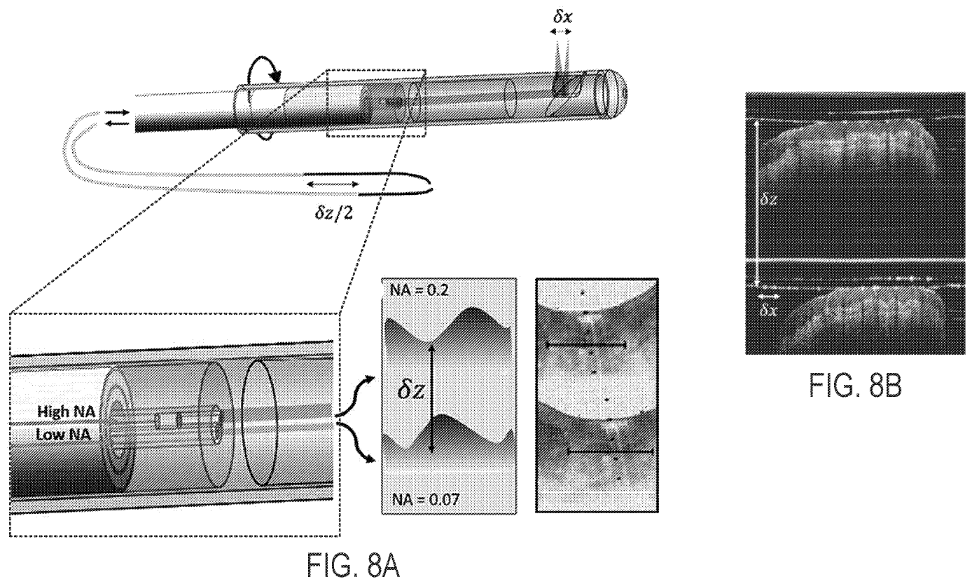

In the two-(or more) optical fiber configuration of FIGS. 1B and 3A-3B, the device contains two optical fibers in the probe device which spin at the same rate and interface with a singular rotary junction. The rotary junction is located proximal to the beam splitter 304 in FIG. 3A so that the rotary junction only needs to accommodate a single fiber; the use of a single fiber for data collection is made possible by the shifting of the two images relative to one another (which in turn is a result of the two beams having different path lengths) so that they can be contained within the same tomogram without overlapping (see FIG. 8B). In a non-limiting example, the two fibers are differentiated in two ways: firstly, they have differently functionalized tips which result in two optical beams of different diameters upon exiting the fibers, and ultimately two images of different resolution after each passes through a common focusing lens. Secondly, they may differ in length, such that based on the interferometric nature of OCT, the cross-sectional image formed by the light traveling out of and back into the longer fiber appears in the same frame as that of the shorter fiber, but below the first image in depth, separated by sufficient distance to make a second independent image copy. Therefore, this realization of the probe device enables the acquisition of two OCT images, simultaneously, at two different resolutions, in a single tomogram.

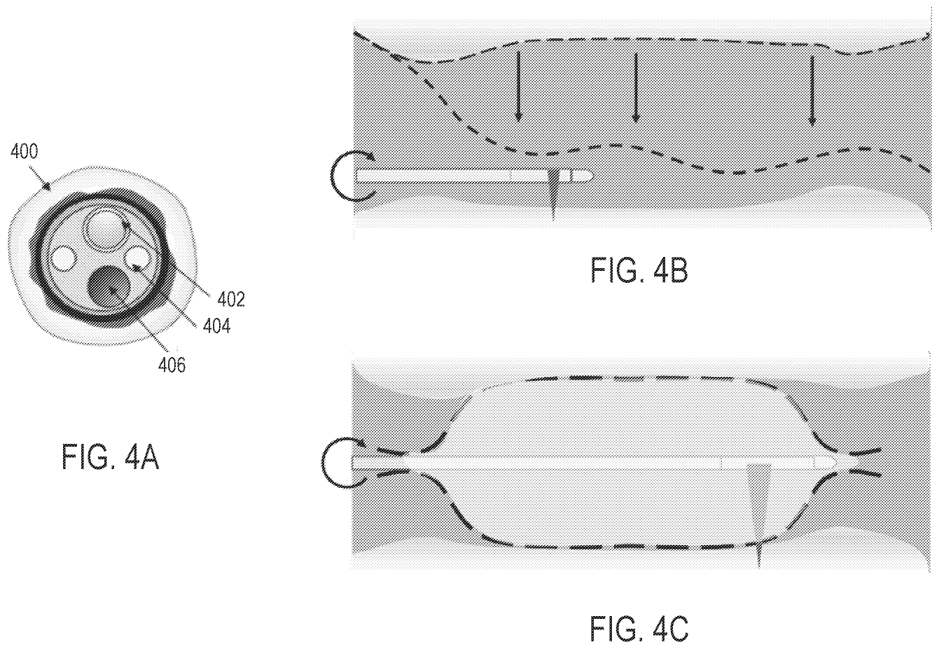

To achieve a target high NA, the sheath surrounding the probe is placed directly in contact with the sample as shown in FIGS. 4A-4C. For clinical endoscopic deployment, the catheter-based probe is introduced to the instrument channel of a standard clinical endoscope and interfaces with the patient through this device. In one non-limiting example shown in FIG. 4A, an endoscope is inserted into the of the esophagus 400, the endoscope featuring a white light channel 402, an instrument channel for introduction of a catheter-based imaging probe 404 as described herein, and a camera 406. The narrow catheter diameter (<2 mm including the sheath) and resulting short distance to the tissue enable the short focal length and high NA of the high-resolution fiber channel. In a non-limiting example, the probe could also be used in conjunction with a distally-inflated balloon in a configuration with optical channels of longer focal length, which would provide a smooth tissue surface and more comprehensive circumferential scanning in tubular organs (FIG. 4C).

In a non-limiting example, the OCT system may be a benchtop or a cart-based console.

Within the console is enclosed a commercial wavelength-swept laser source, which delivers light into the experimental probe. The probe in turn delivers the light to the tissue.

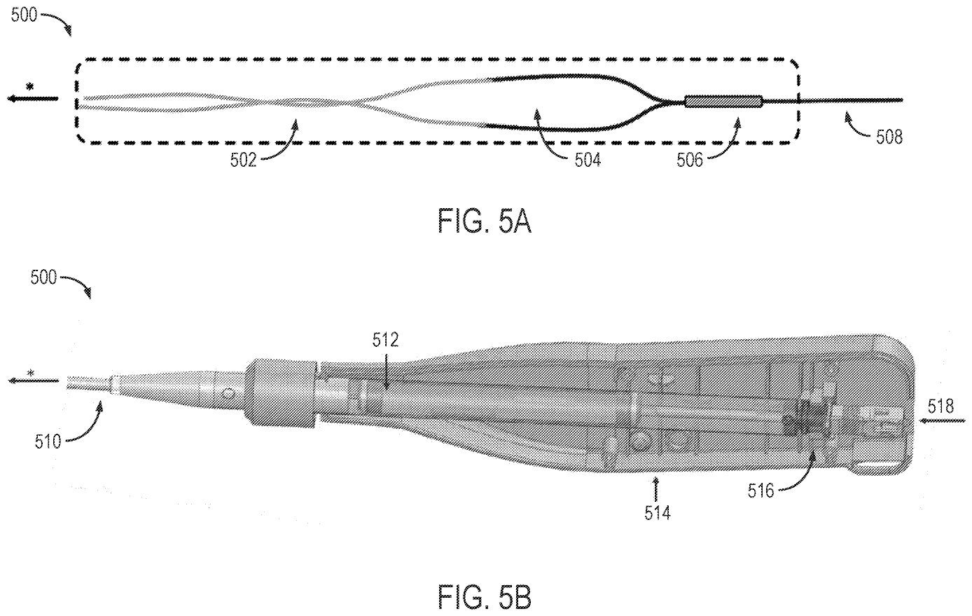

Referring to FIGS. 5A-5B, the probe includes a handle 500 at its proximal end, connecting the probe to the OCT system. In FIG. 5A, the handle 500 is a dual-fiber embodiment features two optical fibers 502 with functionalized tips, spliced to the output ends 504 of a 50/50 fiber-based beam splitter 506. The input fiber to the beam splitter 506 interfaces with the rotary junction 508 and the dashed border shows the enclosure of the fiber within the custom handle 500. In a non-limiting example, the probe connects to the OCT system via the fiber optic rotary junction within the handle 500, which spins/rotates the probe. (Alternatively, the distal reflective element in the probe could be rotated using a micromotor 110 fixed within the probe, as shown in FIG. 1D and discussed above.) As the probe spins, it is able to produce cross-sectional, circumferential images of the sample (e.g., tissue of interest); the probe can further obtain longitudinal volumes if it is also pulled back while it is rotating.

FIG. 5B is a 3D cross-sectional rendering of the probe schematically shown in FIG. 5A with fiber(s) terminating distally in the catheter sheath 510. Furthermore, a tube 512 within the 3D-printed housing 514 holds the beam splitter 506 and interfaces with the rotary junction 516 via a single fiber 518.

More generally, the fiber probe design is not limited to deployment via endoscopy or imaging of the gastrointestinal tract, or to a design implemented by usage of multiple optical fibers as opposed to free-space optical elements. Potential applications include any areas of imaging wherein a catheter-based device would be deployed, such as within the lumen of blood or lymph vessels, and within internal organs, including bladder, gallbladder, lungs, and cervix, and also non-luminal organs such as the brain. Rotation of the catheter-based device could be achieved through either proximal (e.g., rotary junction) or distal (micromotor) scanning hardware, as disclosed herein.

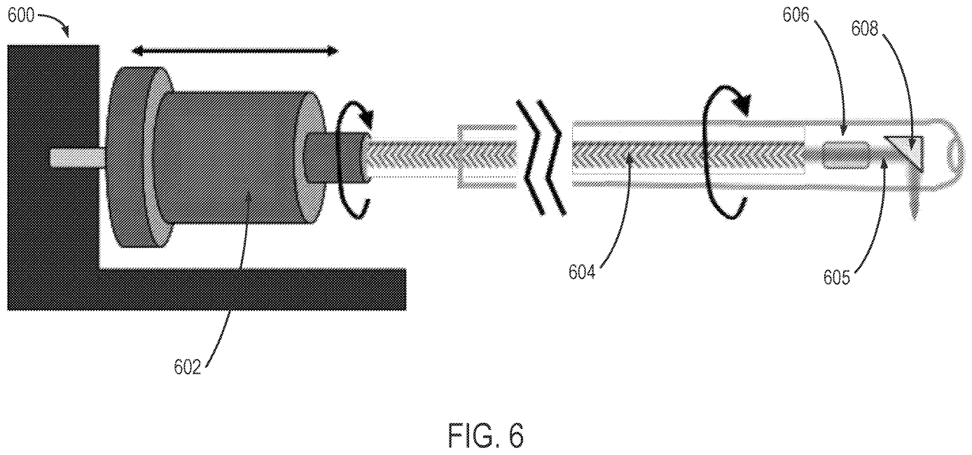

FIG. 6 shows a non-limiting example of the rotary junction and its connection to various components of a handle and probe. The handle 600, similar to the handle shown in FIG. 5B, includes the rotary junction 602. The rotary junction 602 is fixed within the handle 602 and attaches to a driveshaft 604 that extends into the probe and rotates upon actuation of the rotary junction 602. A beam 605 emitted from one or more optical fibers passes through a lens 606 and is reflected into a sample via reflective element 608.

In a non-limiting example, the rotary junction may be controlled using a software routine written to communicate with a microcontroller associated with the rotary junction and securely loaded on the probe. The one or more optical fibers of the probe connect to the rotary junction, allowing them to rotate and pullback with software-controlled actuation of integrated transverse and longitudinal motors. The proximal end of the rotary junction is connected via optical fiber to the interferometer as part of a sample arm.

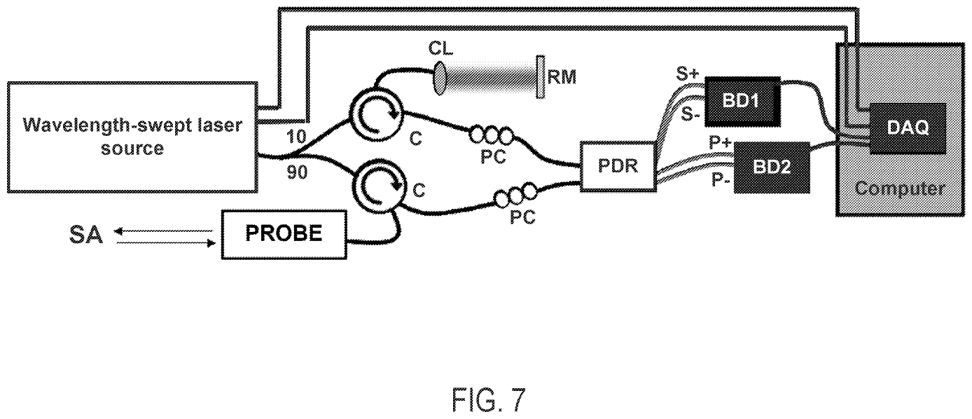

Referring now to FIG. 7, a non-limiting example OCT interferometer system is shown, in various embodiments, other types of OCT interferometer systems may also be used with the disclosed probes and procedures. A standard Mach-Zehnder interferometer-based OCT system is shown. It is important to note that as opposed to other optical microscopy techniques that rely on light propagation in free space, OCT makes use of single-mode fiber components. As a practical advantage, this facilitates the construction of robust and compact systems with high compatibility with space-constrained clinical environments. Here, the light source is coupled to an output optical fiber, which then splits the light to divert most (90%) of the power to form the sample signal, in the interferometer sample arm, and the remaining (10%) power to the reference arm. In various embodiments, the split ratio does not necessarily need to be 90/10, but generally any ratio in which most of the power is going to the sample arm is preferred; in one particular embodiment of an OCT system being used with embodiments of the disclosed probe the split ratio is 92/8, with 92% of the light being directed to the sample arm and 8% being directed to the reference arm. This uneven power split is motivated by the weak backscattering potential of tissue compared to that of the reference mirror (RM), which coherently amplifies the low tissue signal. Fiber-based circulators (C) are used to route light in each arm to either the sample or the reference mirror, respectively, then towards recombination at a fiber-based beam splitter, where the signal interference takes place. After the beam splitter, the optical signal is collected by a photodetector (PDR). The beam splitter has two input channels to take in the reference and sample arm signals, and two output channels. In a non-limiting example, polarization-diverse detection may be employed to split the light into two orthogonal polarization states via a polarization controller (PC), which helps to mitigate tomogram artifacts introduced by any changes the sample has introduced to the polarization state of the light. As shown in FIG. 7, embodiments of the disclosed probe (labeled “PROBE”) are attached at the sample arm (SA) of the OCT interferometer system.

In a non-limiting example, the multi-fiber design also potentiates multiplexed acquisition of signals with other optically-varying parameters, such as optical spectrum, polarization state, angular collection extent, or focal offset to the imaged sample. This design could also be realized using a single-fiber design within an otherwise similar catheter-based OCT system through the usage of distal free-space optical elements. In general, the simultaneous acquisition of depth-multiplexed optical signals with spectral, polarization, angular, or focal variation could also be achieved through reflective components able to alter and separate such signals prior to collection and recording. Through spatial separation by reflective free-space elements, parameter variation may be introduced through introductive coatings, deflecting elements, or similar.

As disclosed herein, images are formed when the one or more fibers, contained within the probe which interfaces with an imaging console through a custom-designed handle, are spun by a single commercial rotary junction or micromotor. In one realization of this device two independent optical fibers are used to create two beams, while another realization of the device could feature a single optical fiber combined with a free-space spatial-multiplexing optical element, such as a diffraction grating or lateral displacement beam splitter, or alternatively, with more than two fibers to collect additional signals. Based on a design originally implemented in free-space in a benchtop OCT system, common information is leveraged in each scan to compensate for the confocal function in both image copies. Further, where multiple scattering is dominating the image, it is inferred by comparing the attenuation coefficients mapped throughout tissue at each resolution, and expect that without the presence of MS, these values will be equivalent (FIG. 2).

With respect to generating the tomogram, FIG. 8A shows the distal tip of a probe device featuring two optical fibers with high and low NA outputs which are fixed parallel to each other and mounted to optical elements to generate two beams which are separated laterally (δx). Proximally, a difference in the lengths of each fiber creates an axial separation of the two output images (δz) in the tomogram (FIG. 8B), with each image corresponding to the signal acquired by each fiber as the probe is rotated with respect to the sample surface. In a non-limiting example, to separate the two image copies in depth (δz) and thus the ability to acquire each simultaneously, the fibers differ in length to create a total separation of, at minimum, 3 mm between each copy, within a maximum imaging range of 11 mm which is dictated by the particular wavelength-swept light source and electronic bandwidth of the custom console.

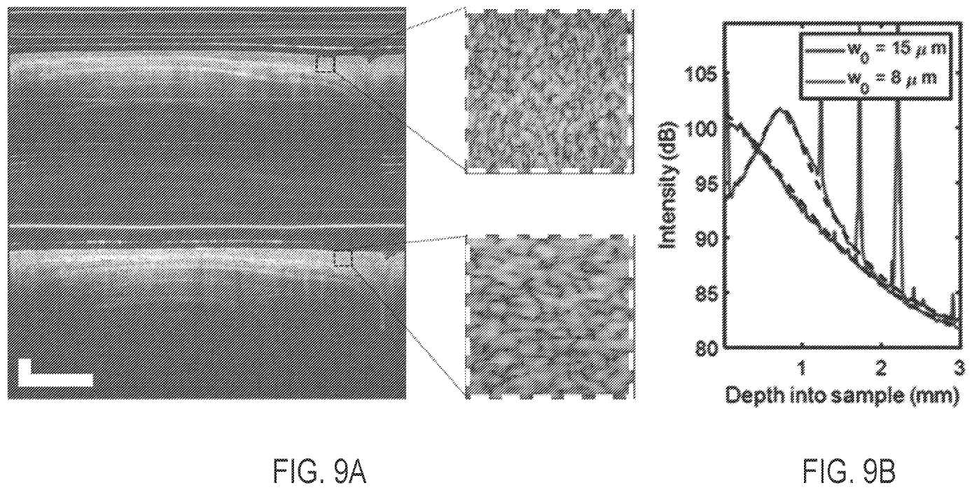

FIGS. 9A-9B show another example of imaging data obtained from a dual-fiber probe as described herein. In this example, a dual-fiber probe was actuated at a rotation rate of 20 revolutions per second to acquire cross-sectional images of a fingertip. A separation distance of 6 mm was achieved between the two image copies. It is evident from the tissue speckle pattern (see FIG. 9A) that the top image copy (from the higher NA fiber) has higher resolution and finer speckle than the lower-resolution bottom image copy (from the lower NA fiber). Separately, in an imaging phantom, intensity decay profiles were fit to a theoretical model to assess the resolution or minimum spot size achievable in each copy (FIG. 9B). In the higher NA copy (NA=0.13), the impact of the location of the focus into the sample is evidenced by the strong peak, whereas this is absent in the lower NA copy (NA=0.07).

The following are non-limiting examples of the development of the system and method implementing that which is described herein, including examples relating to testing of the probe design, signal processing, and image generation.

### EXAMPLES

### Example 1—Methods

Based on measurements of attenuation coefficient (μ) in tissue-mimicking phantoms with a benchtop OCT system, it was determined that to achieve the desired MS-based contrast, one channel of the dual-fiber probe would need an effective NA exceeding 0.1, in addition to one channel offering a resolution comparable to conventional endoscopic OCT systems (NA=~0.05), to offer comparison to more conventional endoscopic OCT imaging. In general, it is expected that more MS-based contrast will be achievable with greater difference in NA; however, given that increasing NA will also degrade the depth of focus in that channel, a maximum value of NA=0.15 was desired. After this point, it is expected that further increasing NA will provide diminishing returns with respect to MS contrast and will primarily serve to deteriorate depth of focus in the high-resolution channel.

In the device prototype realization using two optical fibers within the catheter probe, to customize the two fibers that would produce two different NA (and thus two different image resolutions) while imaging through the same graded refractive index (GRIN) lens, we spliced short lengths of GRIN and coreless fiber to the tips of these fibers in different combinations (FIGS. 3A-3B).

The arrangement of the two fibers in the catheter, wherein each fiber tip is situated parallel to the other and mounted at a fixed distance to the GRIN lens, results in a slight lateral offset between the image copies of the two channels (δx, FIGA. 8A-8B). To achieve the separation of the two image copies in depth (δz) and thus the ability to acquire each simultaneously, the fibers differ in length to create a total separation of, at minimum, 3 mm between each copy, within a maximum imaging as dictated by the wavelength-swept light source and electronic bandwidth of the custom console.

To interface with the commercial rotary junction and actuate the fiber spinning for circumferential image formation (and longitudinal pullback for spirally-acquired volume), a custom handle for the probe was designed at the opposite end of the customized fiber tips (FIG. 5B). The handle houses the fiber-based 50/50 beam splitter, which delivers light to each fiber from the output optical fiber of the imaging console, along with elements for adding mechanical stability. The rotary junction and custom probe handle feature a common optical interface connecting an output fiber from the central system and interferometer with the input fiber of the beam splitter (FIG. 5A).

### Example 2—Image and Signal Processing

Following standard tomogram reconstruction, data were separated by input light polarization state (A-line interleaved signals) and NA (depth-multiplexed signals) for independent treatment. A novel filtering strategy was used to enhance image quality by calculating the mean power spectrum (e.g., lateral frequency content) of complex OCT fringe data to optimally select the width of the spectral window used to process this data. This strategy was particularly useful given that the high Nyquist sampling criterion for the higher NA channel led to significant oversampling in the lower NA channel, since each fiber was subject to the same rotation speed for sample scanning. These optimum filtering methods exploited this oversampling to increase the contrast-to-noise ratio in both channels, but particularly in the lower NA channel. This led to visible improvement in image quality as assessed in the oral mucosa of a healthy volunteer, a stratified squamous epithelial tissue with structure known to be similar to that of the esophagus.

For scattering property analysis, image quality was further improved using TNode computational despeckling. Each NA signal copy was treated separately to finetune despeckling parameters, which require independent optimization for data with different effective lateral resolutions. As previously discussed, despeckling not only improves image quality, but also facilitates our layer-based scattering property calculations in tissue, which require automatic detection of sample layers differentiable by α or μ, and are more challenging to implement in the presence of speckle noise.

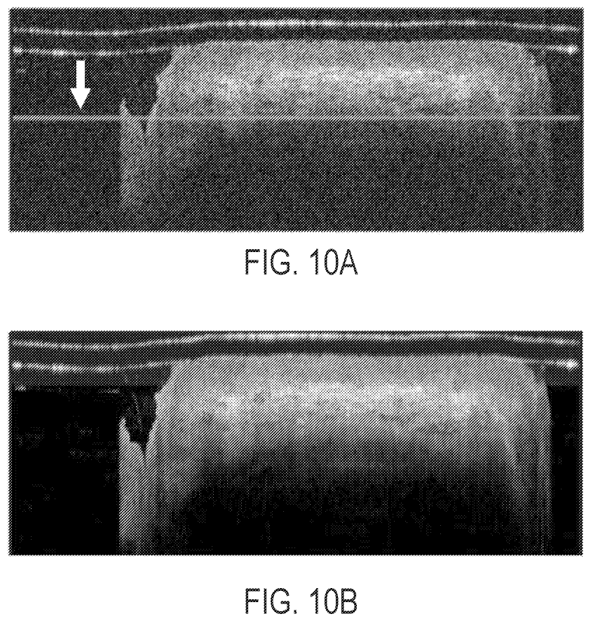

The path of each (higher and lower NA) incident beam from the output facet of the custom fiber tip through multiple air-glass interfaces generated artifacts visible in the tomogram as horizontal lines at constant axial positions, some of which were unavoidably located within the tissue imaging ROIs (FIG. 10A). To prevent these thin yet bright lines from impacting image interpretation or any quantitative analysis, they were removed by an extrapolation routine which replaced artifact intensity values with log-linear intensity based on the values immediately above and below the artifacts prior to converting the signal back to linear scale (FIG. 10B).

### Example 3—Image Registration

In the construction of the dual-fiber catheter-based probe, the two fiber tips were mounted parallel to each other to pass light through a common GRIN lens (FIG. 3A). This produced a small offset in the location of each beam as it was scanned over a sample with driveshaft rotation. For direct comparison of the two differential NA images, an image registration procedure was necessary to account for this offset.

To determine image registration parameters and assess whether the offset in image separation was purely lateral or had an out-of-plane component, a gear-shaped phantom with rotationally distinct features was designed and 3-D printed. The inner diameter of the phantom fit snugly about the catheter probe sheath, and radial features were designed to provide a substrate with a clearly visible ground truth structure for image copy matching. Following imaging and tomogram circularization, direct comparison of the higher and lower NA image copies confirmed the presence of the offset. In MATLAB, the built-in function imregtform was used to determine the appropriate set of volumetric transform parameters, which were then applied to compensate for this offset in differential NA data. The success of this procedure was also confirmed in en face view of the phantom's longitudinal profile. From visualization of en face slabs, it was apparent that the offset was primarily lateral as expected, and successful alignment of the fiber tips had produced minimal angular offset in the signals, enabling a straightforward registration procedure.

### Example 4—Results

Apart from the specific motivations for the usage of this device in endoscopic gastrointestinal imaging detailed above, there are many additional opportunities for this design of multi-fiber imaging probe to be used productively. In terms of device design, the usage of a beam splitter with more than two output fibers, or a free-space spatial multiplexing element interfacing with a single fiber and additional optical modulation elements, would straightforwardly enable the multiplexing of more than two, and potentially several, optical channels. Further, the modulation of the optical parameters of each of these channels is expandable far beyond the modification of NA and image resolution. Other opportunities for optical parameter modulation between channels include optical transmission, polarization state, working distance, spectral behavior, angle of sample illumination, and introduction of aberrations. Through the use of elements within the catheter and imaging console, each of these different forms of contrast could be achieved in conjunction with one another, and even flexibly for a single probe device with the modular exchange of such elements. The interface between the probe and the imaging console could also be modified to enable different paradigms of beam scanning (or lack thereof), such as forward-looking scanning for hollow organ imaging, or rotational scanning at a fixed longitudinal position.

The biomedical applications for OCT imaging with this probe are also not limited to dysplasia detection with the gastrointestinal tract. OCT imaging in hollow organs such as the bladder and cervix could also leverage this design for the detection of dysplastic or other lesions with structural or molecular contrast. The catheter-based design could also benefit intravascular imaging in building upon currently achievable contrast for the optical characterization of coronary arteries or lymphatic vessels. With a multicore fiber design, solid organs such as the brain would also be accessible for imaging. Multicore fiber imaging presents an alternative approach for multi-modality catheter-based imaging. The two approaches could further be combined to span a wide range of probe sizes, functionalization, and applications. Multiparametric OCT imaging is also attractive for other biomedical applications previously explored using catheter-based devices for probe delivery, including characterization of fibrosis in bronchioles within the lungs, pre-cancerous oral lesions, or as a flexible scanning device for dermatologic applications, including skin cancer detection. The probe design is also broadly applicable beyond medical and preclinical clinical and research fields as a sensitive tool for material property characterization in industrial settings.

Our dual-resolution, catheter-based probe for endoscopic OCT acquires imaging data at two different resolutions simultaneously. Several elements of the design benefit from a high degree of customizability, including the resolutions of the two image copies based on the design of each fiber tip or of other free-space distal optical elements for NA modulation, the separation in depth of the two image copies based on the length of each fiber, and the relative signal-to-noise ratio of each channel based on the split ratio of the fiber coupler. The device may improve the accuracy and interpretability of attenuation coefficient imaging in a clinical study and better assess this parameter as a potential biomarker for esophageal dysplasia.

The probe device is believed novel in part because it encloses multiple optical fibers in a catheter to be spun by a single rotary junction to allow the simultaneous collection in vivo of multiple distinct OCT images with varying optical behavior, such as higher and lower resolutions, whereas conventional catheter-based OCT systems utilize just one fiber within the catheter for OCT imaging with fixed optical properties. Other realizations of the device using a single optical fiber with a free-space optical element for distal beam-splitting are also believed to be novel due to this unique hardware configuration that is more flexible (i.e., enables more ways to vary optical contrast between the two channels, such as resolution/NA, spectral properties, polarization, and angular deflection) than other designs making use of collinear beams.

Other strategies exist for multi-channel imaging but utilize multi-channel rotary junctions rather than our scheme for using a standard, single channel rotary junction to spin more than one or more optical fibers, which avoids significant hardware complication, and to the best of our knowledge, multi-channel endoscopic OCT image acquisition has not been previously deployed to simultaneously collect images at different resolutions, or to achieve other means of optical parameter modulation of an OCT signal.

It will be appreciated by those skilled in the art that while the disclosed subject matter is described above in connection with particular embodiments and examples, the invention is not necessarily so limited, and that numerous other embodiments, examples, uses, modifications and departures from the embodiments, examples and uses are intended to be encompassed by the claims attached hereto. Each reference cited herein is incorporated by reference in its entirety.

Various features and advantages of the invention are set forth in the following claims.

What is claimed is:

1. An apparatus for multi-parametric optical coherence tomography (OCT), comprising:
an imaging system;
a probe coupled to the imaging system and including a proximal end and a distal end, the probe comprising:
at least one optical fiber including a fiber proximal end and a fiber distal end and disposed within the probe, the fiber distal end extending at least a partial length between the proximal end and the distal end of the probe, and
a plurality of optical elements positioned at the distal end of the probe and positioned distal to the fiber distal end; and
a processor coupled to the imaging system and configured to:
direct at least two spatially-offset light beams at a sample,
collect data returned from the sample from the at least two light beams through the probe, combine the data, and
analyze the combined data to determine at least one sample parameter.

2. The apparatus of claim 1, wherein the plurality of optical elements includes a lens and a reflective element configured to direct a beam from the at least one optical fiber into a sample, wherein the reflective element is disposed distal to the lens.

3. The apparatus of claim 2, wherein the lens is a graded refractive index (GRIN) lens.

4. The apparatus of claim 2, wherein the reflective element is a mirror.

5. The apparatus of claim 2, wherein the plurality of optical elements further includes a diffractive optical element.

6. The apparatus of claim 5, wherein the diffractive optical element includes a beam splitter.

7. The apparatus of any one of claims 1-6, wherein the at least two spatially-offset light beams comprises a first light beam having a first numerical aperture (NA₁) and a second light beam having a second numerical aperture (NA₂) different from the first numerical aperture (NA₁).

8. The apparatus of claim 7, wherein the first numerical aperture is higher than the second numerical aperture.

9. The apparatus of claim 7, wherein the data comprises a first tomogram comprising data from the first beam and a second tomogram comprising data from the second beam.

10. The apparatus of claim 9, wherein the first tomogram and the second tomogram comprise a single tomogram.

11. The apparatus of claim 9, wherein the processor, when combining the data, is further configured to:
subtract noise from the first tomogram and the second tomogram,
separate the first tomogram and second tomogram,
compensate for a confocal function of the imaging system, and
de-speckle the first tomogram and the second tomogram.

12. The apparatus of claim 9, wherein the imaging system comprises an optical interferometric system.

13. The apparatus of claim 12, wherein the optical interferometric system comprises a spectral domain optical coherence tomography (SD-OCT) system or an optical frequency domain imaging (OFDI) system, and
wherein the probe is an OCT probe.

14. The apparatus of claim 13, wherein the OCT probe is a rotary probe coupled to the optical interferometric system by a rotary junction, and
wherein the processor, when collecting the data, is further configured to:
cause the OCT probe to rotate,
collect the data from the optical interferometric system at a plurality of radial positions, and
generate a cross-sectional image of the sample based on collecting data from the optical interferometric system at the plurality of radial positions.

15. The apparatus of claim 14, wherein the processor, when analyzing the data, is further configured to:
calculate attenuation coefficients (μ) for each of the first tomogram and the second tomogram, and
determine a degree of multiple scattering in the sample based on calculating the attenuation coefficients.

16. The apparatus of claim 1, wherein the probe includes at least two optical fibers.

17. The apparatus of claim 16, wherein the at least two optical fibers differ from one another in at least one of: output beam diameter, fiber length, or type of functional tip at the fiber distal end.

18. The apparatus of claim 16, wherein the at least two optical fibers includes a first optical fiber and a second optical fiber,
wherein the first optical fiber is adjacent to the second optical fiber,
wherein the at least two spatially-offset light beams comprise a first light beam and a second light beam, and
wherein the first optical fiber emits the first light beam having a first numerical aperture and
wherein the second optical fiber emits the second light beam having a second numerical aperture different from the first numerical aperture.

19. The apparatus of claim 18, wherein the first optical fiber comprises a first optical element coupled to a distal end thereof and configured to emit the first light beam with the first numerical aperture, and
wherein the second optical fiber comprises a second optical element coupled to a distal end thereof and configured to emit the second light beam with the second numerical aperture.

20. The apparatus of claim 18, wherein the first optical fiber and the second optical fiber are different lengths.

21. The apparatus of claim 20, wherein the processor, when directing at least two spatially-offset light beams at the sample, is further configured to:
direct the first light beam from the first optical fiber at the sample and direct the second light beam from the second optical fiber at the sample,
wherein the processor, when collecting data returned from the sample, is further configured to:
collect data returned from the sample based on the first light beam and the second light beam,
wherein the processor, when combining the data, is further configured to:
combine the data returned from the sample based on the first light beam and the second light beam to form a single tomogram.

22. The apparatus of claim 1, wherein the processor, when combining the data, is further configured to:
combine the data to provide a depth-multiplexed optical signal based on at least one of a spectral, polarization, angular, or focal variation between the at least two spatially-offset light beams.

23. The apparatus of claim 1, wherein the plurality of optical elements comprises a free-space spatial multiplexing element.

24. The apparatus of claim 23, wherein the free-space spatial multiplexing element is configured to split a light beam into the at least two spatially-offset light beams.

25. The apparatus of claim 24, wherein a first path length of a first light beam of the at least two spatially-offset light beams is shorter than a second path length of a second light beam of the at least two spatially-offset light beams.

26. The apparatus of claim 24, wherein a first numerical aperture of a first light beam of the at least two spatially-offset light beams is higher than a second numerical aperture of a second light beam of the at least two spatially-offset light beams.

27. The apparatus of claim 23, wherein the free-space spatial multiplexing element comprises at least one of a lateral beam splitter or a diffraction grating combined with a lens.

28. A method for multi-parametric optical coherence tomography (OCT), the method comprising:
providing a probe coupled to an imaging system, the probe including a proximal end and a distal end, the probe comprising:
at least one optical fiber including a fiber proximal end and a fiber distal end and disposed within the probe, the fiber distal end extending at least a partial length between the proximal end and the distal end of the probe, and
a plurality of optical elements positioned at the distal end of the probe and positioned distal to the fiber distal end; and
directing, using a processor coupled to the imaging system, at least two spatially-offset light beams at a sample,
collecting, using the processor, data returned from the sample from the at least two light beams through the probe,
combining, using the processor, the data, and
analyzing, using the processor, the combined data to determine at least one sample parameter.

29. The method of claim 28, wherein the plurality of optical elements includes a lens and a reflective element,
wherein the reflective element is disposed distal to the lens, and
wherein the method further comprises:
directing a beam from the at least one optical fiber into a sample via the lens and the reflective element.

30. The method of claim 29, wherein the lens is a graded refractive index (GRIN) lens.

31. The method of claim 29, wherein the reflective element is a mirror.

32. The method of claim 29, wherein the plurality of optical elements further includes a diffractive optical element.

33. The method of claim 32, wherein the diffractive optical element includes a beam splitter.

34. The method of any one of claims 28-33, wherein directing at least two spatially-offset light beams at a sample further comprises:
directing at least two spatially-offset light beams at a sample,
wherein the at least two spatially-offset light beams comprises a first light beam having a first numerical aperture (NA₁) and a second light beam having a second numerical aperture (NA₂) different from the first numerical aperture (NA₁).

35. The method of claim 34, wherein the first numerical aperture is higher than the second numerical aperture.

36. The method of claim 34, wherein collecting data further comprises:
collecting a first tomogram comprising data from the first beam and a second tomogram comprising data from the second beam.

37. The method of claim 36, wherein the first tomogram and the second tomogram comprise a single tomogram.

38. The method of claim 36, wherein combining the data further comprises:
subtracting noise from the first tomogram and the second tomogram,
separating the first tomogram and second tomogram,
compensating for a confocal function of the imaging system, and
de-speckling the first tomogram and the second tomogram.

39. The method of claim 36, wherein providing a probe coupled to an imaging system further comprises:
providing the probe coupled to the imaging system,
wherein the imaging system comprises an optical interferometric system.

40. The method of claim 39, wherein the optical interferometric system comprises a spectral domain optical coherence tomography (SD-OCT) system or an optical frequency domain imaging (OFDI) system, and
wherein the probe is an OCT probe.

41. The method of claim 40, wherein the OCT probe is a rotary probe coupled to the optical interferometric system by a rotary junction, and
wherein collecting the data further comprises:
causing the OCT probe to rotate,
collecting the data from the optical interferometric system at a plurality of radial positions, and
generating a cross-sectional image of the sample based on collecting data from the optical interferometric system at the plurality of radial positions.

42. The method of claim 41, wherein analyzing the data further comprises:
calculating attenuation coefficients (μ) for each of the first tomogram and the second tomogram, and
determining a degree of multiple scattering in the sample based on calculating the attenuation coefficients.

43. The method of claim 28, wherein the probe includes at least two optical fibers.

44. The method of claim 43, wherein the at least two optical fibers differ from one another in at least one of: output beam diameter, fiber length, or type of functional tip at the fiber distal end.

45. The method of claim 43, wherein the at least two optical fibers includes a first optical fiber and a second optical fiber,
wherein the first optical fiber is adjacent to the second optical fiber,
wherein the at least two spatially-offset light beams comprise a first light beam and a second light beam, and
wherein the method further comprises:
emitting the first light beam having a first numerical aperture from the first optical fiber, and
emitting the second light beam having a second numerical aperture from the second optical fiber, wherein the second numerical aperture is different from the first numerical aperture.

46. The method of claim 45, wherein the first optical fiber comprises a first optical element coupled to a distal end thereof,
wherein the second optical fiber comprises a second optical element coupled to a distal end thereof, and
wherein the method further comprises:
emitting the first light beam with the first numerical aperture from the first optical element, and
emitting the second light beam with the second numerical aperture from the second optical element.

47. The method of claim 45, wherein the first optical fiber and the second optical fiber are different lengths.

48. The method of claim 47, wherein directing at least two spatially-offset light beams at the sample further comprises:
directing the first light beam from the first optical fiber at the sample and directing the second light beam from the second optical fiber at the sample,
wherein collecting data returned from the sample further comprises:
collecting data returned from the sample based on the first light beam and the second light beam, and
wherein combining the data further comprises:
combining the data returned from the sample based on the first light beam and the second light beam to form a single tomogram.

49. The method of claim 28, wherein combining the data further comprises:
combining the data to provide a depth-multiplexed optical signal based on at least one of a spectral, polarization, angular, or focal variation between the at least two spatially-offset light beams.

50. The method of claim 28, wherein the plurality of optical elements comprises a free-space spatial multiplexing element.

51. The method of claim 50, further comprising:
splitting a light beam into the at least two spatially-offset light beams using the free-space spatial multiplexing element.

52. The method of claim 51, wherein a first path length of a first light beam of the at least two spatially-offset light beams is shorter than a second path length of a second light beam of the at least two spatially-offset light beams.

53. The method of claim 51, wherein a first numerical aperture of a first light beam of the at least two spatially-offset light beams is higher than a second numerical aperture of a second light beam of the at least two spatially-offset light beams.

54. The method of claim 50, wherein the free-space spatial multiplexing element comprises at least one of a lateral beam splitter or a diffraction grating combined with a lens.

55. An apparatus for multi-parametric optical coherence tomography (OCT), comprising:
a probe including a proximal end and a distal end, the probe comprising:
at least one optical fiber including a fiber proximal end and a fiber distal end and disposed within the probe, the fiber distal end extending at least a partial length between the proximal end and the distal end of the probe, and
a plurality of optical elements positioned at the distal end of the probe and positioned distal to the fiber distal end,
the probe being configured to:
direct at least two spatially-offset light beams at a sample, and
receive image information returned from the sample from the at least two light beams through the probe.

56. The apparatus of claim 55, wherein the plurality of optical elements includes a lens and a reflective element configured to direct a beam from the at least one optical fiber into a sample, wherein the reflective element is disposed distal to the lens.

57. The apparatus of claim 56, wherein the lens is a graded refractive index (GRIN) lens.

58. The apparatus of claim 56, wherein the reflective element is a mirror.

59. The apparatus of claim 56, wherein the plurality of optical elements further includes a diffractive optical element.

60. The apparatus of claim 59, wherein the diffractive optical element includes a beam splitter.

61. The apparatus of any one of claims 55-60, wherein the at least two spatially-offset light beams comprises a first light beam having a first numerical aperture (NA₁) and a second light beam having a second numerical aperture (NA₂) different from the first numerical aperture (NA₁).

62. The apparatus of claim 61, wherein the first numerical aperture is higher than the second numerical aperture.

63. The apparatus of claim 61, wherein the image information comprises a first tomogram comprising image information from the first beam and a second tomogram comprising image information from the second beam.

64. The apparatus of claim 63, wherein the first tomogram and the second tomogram comprise a single tomogram.

65. The apparatus of claim 64, wherein the probe is an OCT probe.

66. The apparatus of claim 55, wherein the probe includes at least two optical fibers.

67. The apparatus of claim 66, wherein the at least two optical fibers differ from one another in at least one of: output beam diameter, fiber length, or type of functional tip at the fiber distal end.

68. The apparatus of claim 66, wherein the at least two optical fibers includes a first optical fiber and a second optical fiber,
wherein the first optical fiber is adjacent to the second optical fiber,
wherein the at least two spatially-offset light beams comprise a first light beam and a second light beam, and
wherein the first optical fiber emits the first light beam having a first numerical aperture and wherein the second optical fiber emits the second light beam having a second numerical aperture different from the first numerical aperture.

69. The apparatus of claim 68, wherein the first optical fiber comprises a first optical element coupled to a distal end thereof and configured to emit the first light beam with the first numerical aperture, and
wherein the second optical fiber comprises a second optical element coupled to a distal end thereof configured to emit the second light beam with the second numerical aperture.

70. The apparatus of claim 68, wherein the first optical fiber and the second optical fiber are different lengths.

71. The apparatus of claim 70, wherein the probe, when directing at least two spatially-offset light beams at the sample, is further configured to:
direct the first light beam from the first optical fiber at the sample and direct the second light beam from the second optical fiber at the sample,
wherein the probe, when receiving image information returned from the sample, is further configured to:
collect image information returned from the sample based on the first light beam and the second light beam.

72. The apparatus of claim 55, wherein the plurality of optical elements comprises a free-space spatial multiplexing element.

73. The apparatus of claim 72, wherein the free-space spatial multiplexing element is configured to split a light beam into the at least two spatially-offset light beams.

74. The apparatus of claim 73, wherein a first path length of a first light beam of the at least two spatially-offset light beams is shorter than a second path length of a second light beam of the at least two spatially-offset light beams.

75. The apparatus of claim 73, wherein a first numerical aperture of a first light beam of the at least two spatially-offset light beams is higher than a second numerical aperture of a second light beam of the at least two spatially-offset light beams.

76. The apparatus of claim 72, wherein the free-space spatial multiplexing element comprises at least one of a lateral beam splitter or a diffraction grating combined with a lens.
